(19) United States
(12) Patent Application Publication
(10) Pub. No.: US 2010/0312083 A1
(43) Pub. Date: Dec. 9, 2010

(54) System for Monitoring Glucose and Measuring Wattage

(76) Inventor: Phil Southerland, Atlanta, GA (US)

Correspondence Address: LUEDEKA, NEELY & GRAHAM, P.C., P O BOX 1871, KNOXVILLE, TN, 37901, US

(21) Appl. No.: 12/762,462

(22) Filed: Apr. 19, 2010

Related U.S. Application Data
(60) Provisional application No. 61/170,711, filed on Apr. 20, 2009.

(51) Int. Cl. A61B 5/145; A61B 5/103

(52) U.S. Cl. 600/365; 600/592

(57) ABSTRACT

A computerized system for monitoring information related to the power being generated by a user and blood glucose levels of the user, the system comprising: a shoe worn by the user, an electronic force sensor associated with the shoe, and a computer electronically coupled to the force sensor.

### CROSS-REFERENCE TO RELATED APPLICATIONS

This application claims priority under all applicable rules and statutes to U.S. Provisional Application Ser. No. 61/170,711, filed Apr. 20, 2009, and entitled “System For Monitoring Glucose and Measuring Wattage,” incorporated herein by reference in its entirety.

### FIELD

This disclosure relates to the field of performance meters. More particularly, this disclosure relates to a system for monitoring power output and blood glucose levels of a user and to utilize the monitored information to help the user maximize their athletic performance.

### BACKGROUND

Long distance cycling and running are two examples of endurance sports. Many times a participant will overly exert themselves at some point during a race and will have diminished performance during later stages of the race, often referred to as “bonking.” Other times, a participant, fearing he may “bonk,” may under exert. In either event, had the participant been able to maintain a suitably high exertion level without under-exerting or reaching an over-exerted state, the participant would likely have performed better in the race.

Another factor that weighs into an athlete's performance is blood glucose and the intake of calories during a race. In the case of athletes having diabetes, blood glucose levels can be even more of a factor in not only performance levels, but also safety.

The present disclosure advantageously provides a system for monitoring power output and blood glucose levels of a user. The system also enables the monitored information to be utilized to help the user maximize their athletic performance to maintain a suitably high exertion level while avoiding under-exerted or over-exerted states.

### SUMMARY

The above and other needs are met by a computerized system for monitoring information related to the power being generated by a user and blood glucose levels of the user.

The system includes a shoe worn by the user, an electronic force sensor associated with the shoe, and a computer electronically coupled to the force sensor.

The system is particularly suited for incorporation into running and cycling shoes, with a sensor in each shoe of the user.

### DETAILED DESCRIPTION

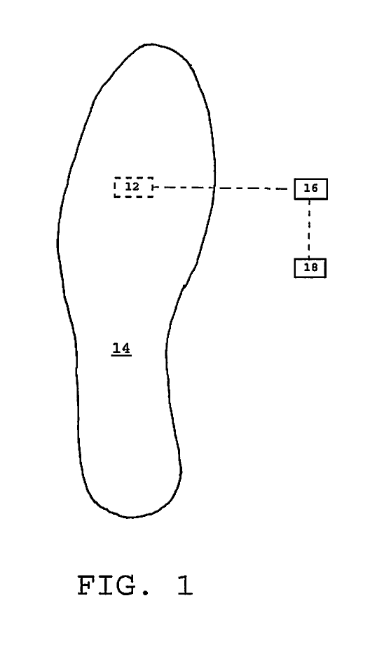

With reference to FIG. 1, there is shown a schematic view of a computerized system 10 that is particularly suitable for use by a sports user, such as cyclists and runners, to monitor information related to the power being generated by the user and also to measure blood glucose level of the user. The system is preferably configured to be incorporated within a shoe worn by the user. Wattage information is very useful to athletes, such as a cyclist, for maintaining a desired exertion level. It has been observed that a correlation exists of wattage relative to the blood glucose level of an athlete.

The system is particularly suitable for use by persons having diabetes, but it will be appreciated that persons not having diabetes may also be able to benefit and optimize their performance. In this regard, the system further utilizes software for evaluating the optimum blood glucose level of the user and further feedback to provide information suitable for assisting in maintaining the blood glucose of the cyclist at a level suitable for peak performance. For example, the software and information concerning the user and the user's exertion level may be used to provide a desired caloric intake for the user. When used by a person having diabetes and using insulin, the system may further enable determination of a desired insulin regimen (times and amounts) for maintaining peak efficiency and power during exercise.

The system 10 includes an electronic force sensor 12 preferably positioned within a shoe sole 14, such as the sole of a running or cycling shoe, and electronically coupled, preferably by wireless transmission, to a computer 16 configured to receive and display the sensed force. The force sensor 12 may advantageously be positioned to lie under the ball of the foot of the user for running and cycling sports.

The sensor 12 is configured to measure force applied by the foot of a user during a running step or a cycling pedal stroke. The system 10 preferably includes at least one of the sensors 12 in each shoe of the user.

The computer 16 may utilize wattage components of the type used in conventional micro-computers of the type used on bicycles to measure wattage, such as power meters marketed under the brand CycleOps power meters, and may be mounted on the bike or worn by the user such as on the wrist. Other measured information may include cadence, speed, and other parameters associated with a runner's strides or a cyclists pedal strokes.

In addition, the computer 16 will also preferably receive signals from a blood glucose sensor 18 associated with the athlete for monitoring blood glucose on a continuous or periodic basis. An example of a continuous blood glucose monitor having a suitable sensor 18 is a monitor available under the tradename Freestyle Navigator.

The computer 16 preferably has various displays, providing information on one display such as wattage, speed, cadence, ride time, blood glucose, and blood glucose trend. Other displays may include information such as graphs of wattage and blood glucose from the previous hour, maximum wattage, and the like. The computer 16 may also include alarms that sound when certain parameters are observed, such as blood glucose levels below about 80 and above about 120 mg/dl.

The computer 16 may also receive information from a heart monitor, such as heart rate, blood pressure, and the like. This information may be used in various combinations to maintain a desired exertion level as well as optimizing performance, such as measuring stride or pedal efficiency, maintenance of desired cadence, and the like. Also, as described above, the wattage information is useful for maintaining a desired exertion level, and is preferably correlated relative to the blood glucose level of an athlete.

The foregoing description of preferred embodiments for this disclosure has been presented for purposes of illustration and description. It is not intended to be exhaustive or to limit the disclosure to the precise form disclosed. Obvious modifications or variations are possible in light of the above teachings. The embodiments are chosen and described in an effort to provide the best illustrations of the principles of the disclosure and its practical application, and to thereby enable one of ordinary skill in the art to utilize the disclosure in various embodiments and with various modifications as are suited to the particular use contemplated. All such modifications and variations are within the scope of the disclosure as determined by the appended claims when interpreted in accordance with the breadth to which they are fairly, legally, and equitably entitled.

What is claimed is:

1. A computerized system for monitoring information related to the power being generated by a user and blood glucose levels of the user, the system comprising: a shoe worn by the user, an electronic force sensor associated with the shoe, and a computer electronically coupled to the force sensor.

2. The system of claim 1, wherein the force sensor is configured to measure force applied by a foot of a user within the shoe.

3. The system of claim 1, further comprising a glucose monitor configured for generating signals associated with blood glucose of the user, the glucose monitor being electronically coupled to the computer, wherein the computer processes the signals corresponding to the blood glucose and the force signals relative to time, and correlates the force and the blood glucose relative to one another.
